(19) United States
(12) Patent Application Publication
(10) Pub. No.: US 2013/0186405 A1
(43) Pub. Date: Jul. 25, 2013

(54) SYSTEM AND METHOD FOR MONITORING MEDICAL EQUIPMENT

(71) Applicants: PAUL KRZYZANOWSKI, FLEMINGTON, NJ (US); DAVID LEEDS, LAKE WORTH, FL (US)

(72) Inventors: PAUL KRZYZANOWSKI, FLEMINGTON, NJ (US); DAVID LEEDS, LAKE WORTH, FL (US)

(73) Assignee: OPENPEAK INC., Boca Raton, FL (US)

(21) Appl. No.: 13/358,508

(22) Filed: Jan. 25, 2012

(51) Int. Cl. A61M 16/06; G01R 19/00; G06F 19/00; G01D 21/00

(52) U.S. Cl. 128/206.21; 73/865.8; 324/76.11; 702/1; 702/64

(57) ABSTRACT

A method and system for monitoring a medical device to ensure usage compliance by a patient are described herein. The method can include the steps of monitoring one or more parameters associated with the operation of the medical device, comparing the monitored parameters with one or more predetermined operational profiles and generating a signal that is indicative of whether the medical device is being used by the patient in substantial conformance with a predetermined usage pattern assigned to that patient. In one arrangement, the use of the medical device by the patient requires voluntary actions on the part of the patient to be in substantial conformance with the predetermined usage pattern.

### FIELD OF TECHNOLOGY

The present subject matter relates to systems and methods for monitoring medical equipment.

### BACKGROUND

Many patients who are afflicted with certain medical conditions must rely on medical equipment to maintain or improve their health. While many of these devices are used in hospitals or other health care settings, some equipment may be operated in the home of a patient. Much of this equipment is paid for by health insurance companies or government agencies. To ensure maximum return on health care related expenditures, such entities may wish to monitor the usage of medical equipment that has been placed in a patient's home. If a patient fails to follow through on a course of treatment, the insurance company or government agency may spend additional dollars on unnecessary care.

### SUMMARY

A method for monitoring a medical device to ensure usage compliance by a patient is described herein. The method can include the steps of monitoring one or more parameters associated with the operation of the medical device, comparing the monitored parameters with one or more predetermined operational profiles and generating a signal that is indicative of whether the medical device is being used by the patient in substantial conformance with a predetermined usage pattern assigned to that patient. In one arrangement, the use of the medical device by the patient requires voluntary actions on the part of the patient to be in substantial conformance with the predetermined usage pattern. The method can also include the step of communicating the signal indicative of whether the medical device is being used by the patient in substantial conformance with the predetermined usage pattern to a remote location. The method can further include the step of maintaining a log that contains information relating to the monitoring of the parameters.

As an example, the monitored parameters can include an on/off state, current (or amperes), volts (or voltage), real power or apparent power. As another example, the predetermined operational profiles can include a predetermined on/off state, a predetermined value or range of values of amperes, a predetermined value or range of values of voltage, a predetermined value or range of values of real power or a predetermined value or range of values of apparent power.

In one arrangement, the medical device can be a breathing assistance machine. Also, the steps of monitoring the parameters, comparing the monitored parameters and generating the signal can be performed at a power device that provides power to the medical device. In another arrangement, the power device can be separate from the medical device.

A medical device monitor is also described herein. The medical device monitor can include a monitoring module that can be operable to monitor one or more parameters associated with the operation of a medical device and a table that can store predetermined operational parameters. The medical device monitor can also include a processing unit that is communicatively coupled to the monitoring module and the table. The processing unit can be configured to compare the monitored parameters with the predetermined operational parameters stored in the table and to generate a signal that is indicative of whether the medical device is being used by a patient in substantial conformance with a predetermined usage pattern assigned to that patient. The processing unit can be further configured to maintain a log that contains information relating to the monitoring of the parameters. The medical device monitor can also include a communication module that can be configured to communicate the signal indicative of whether the medical device is being used by the patient in substantial conformance with the predetermined usage pattern to a remote location.

In one arrangement, the use of the medical device by the patient may require voluntary actions on the part of the patient to be in substantial conformance with the predetermined usage pattern. As an example, the parameters that are monitored by the monitoring module include an on/off state, amperes, voltage, real power or apparent power. As another example, the predetermined operational parameters may include a predetermined on/off state, a predetermined value or range of value of amperes, a predetermined value or range of values of voltage, a predetermined value or range of values of real power or a predetermined value or range of values of apparent power.

In one arrangement, the medical device can be a breathing assistance machine. In addition, the medical device monitor can include a plug that is designed to couple to a power outlet to permit the medical device monitor to provide power to the medical device.

Another medical device monitor to ensure compliance with a prescribed therapy assigned to a patient is described herein. The medical device monitor can include a monitoring module that may be operable to monitor one or more parameters associated with the operation of a medical device, and the medical device may form part of the prescribed therapy. Forming part of the prescribed therapy means that the medical device is intended to be used as part of a therapy that has been prescribed to a patient. The medical device monitor can also include a processing unit that can be coupled to the monitoring module. The processing unit may be configured to generate a signal that is indicative of whether the medical device is being used by the patient in substantial conformance with the prescribed therapy. The medical device monitor can also include a power module that can be configured to provide power to the medical device.

As an example, the medical device monitor can be a component that is separate from the medical device. As another example, the medical device can be a legacy device, and the medical device monitor can be a retrofitted component that can increase the functionality of the legacy medical device.

A medical device monitoring system for ensuring compliance with a prescribed therapy assigned to a patient is also described herein. The system can include a medical device that can form part of the prescribed therapy and a medical device monitor that can provide power to the medical device. The medical device monitor can include a monitoring module that can be configured to monitor one or more parameters associated with the operation of the medical device, a table that can be configured to store predetermined operational parameters and a processing unit. The processing unit can be communicatively coupled to the monitoring module and the table and can be configured to compare the monitored parameters with the predetermined operational parameters stored in the table. The processing unit can also be operable to generate a signal that can be indicative of whether the medical device is being used by the patient in substantial conformance with the prescribed therapy assigned to that patient. As an example, the medical device can be a breathing assistance machine that includes an air mask, and the prescribed therapy may require that the patient wear the air mask.

Another medical device monitoring system that forms part of a prescribed therapy for a patient is described herein. The system can include an air mask that is configured to engage the face of a patient to permit forced air to be provided to the patient. When engaged with the patient, an air pressure can be generated by the air mask. The system can also include a detection module that can be configured to monitor one or more thresholds to determine whether the air mask is engaged with the face of the patient. The system can further include a processing unit that can be configured to generate a signal that is indicative of whether the patient is in substantial conformance with the prescribed therapy based on the frequency that the air mask is engaged with the face of the patient.

The system can further include a communications module that is configured to transmit the generated signal to a remote location. As an example, one of the thresholds used to determine whether the air mask is engaged with the face of the patient is the air pressure generated by the air mask.

A method for determining whether a patient is in substantial conformance with a predetermined usage pattern assigned to that patient is also described herein. The method can include the steps of measuring an air pressure that is generated by an air mask that is intended to be engaged with the face of the patient as part of the predetermined usage pattern and comparing the measured air pressure with a predetermined threshold to determine whether the air mask is engaged with the face of the patient. The method can further include the step of generating a signal that is indicative of whether the patient is in substantial conformance with the predetermined usage pattern based on the frequency that the air mask is engaged with the face of the patient.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the present invention and, together with the description, further serve to explain the principles of the invention and to enable a person skilled in the relevant art(s) to make and use the invention.

FIG. 1 illustrates an example of a medical device monitoring system.

FIG. 2 illustrates an example of a method for monitoring a medical device to ensure usage compliance by a patient.

FIG. 3 illustrates another example of a medical device monitoring system.

Applicants expressly disclaim any rights to any third-party trademarks or copyrighted images included in the figures. Such marks and images have been included for illustrative purposes only and constitute the sole property of their respective owners.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

The following detailed description refers to the accompanying drawings that illustrate exemplary embodiments; however, the scope of the present claims is not limited to these embodiments. Thus, embodiments beyond those shown in the accompanying drawings, such as modified versions of the illustrated embodiments, may nevertheless be encompassed by the present claims.

References in the specification to “one embodiment,” “an embodiment,” “an example embodiment,” or the like, indicate that the embodiment described may include a particular feature, structure, or characteristic, but every embodiment may not necessarily include the particular feature, structure, or characteristic. Moreover, such phrases are not necessarily referring to the same embodiment. Furthermore, when a particular feature, structure, or characteristic is described in connection with an embodiment, it is submitted that it is within the knowledge of one skilled in the art to implement such feature, structure, or characteristic in connection with other embodiments whether or not explicitly described.

Several definitions that apply throughout this document will now be presented. The term “exemplary” as used herein is defined as an example or an instance of an object, apparatus, system, entity, composition, method, step or process. The term “communicatively coupled” is defined as a state in which two or more components are connected such that communication signals are able to be exchanged between the components on a unidirectional or bidirectional (or multi-directional) manner, either wirelessly, through a wired connection or a combination of both. An “electronic device” is defined as a powered component that is configured to perform some process or function for a user.

The term “medical device” is defined as a device that is designed to improve, maintain or monitor the health of a patient or otherwise exhibits one or more curative, medicinal or therapeutic properties. A “patient” is defined as a living being that is under medical care or is otherwise receiving some form of treatment. The term “power device” or “power module” is defined as a component or group of components that provide electrical power to another component or group of components. An “interface” is defined as a component or group of components that at least receive(s) signals from a first device and transfers those signals to a second device in a form that is compatible with the second device.

A “processing unit” is defined as one or more components that execute(s) sets of instructions. The term “table” is defined as a component or group of components that store(s) data for later retrieval. The term “communication module” is defined as a component or a group of components that enable(s) signals to be transmitted or received (or both) over a wired or wireless (or both) medium. The term “breathing assistance machine” is defined as a machine that enables or at least partially supports or otherwise provides some therapeutic benefit to a patient's breathing. An “air mask” is defined as a mask that is configured to engage the face of a patient and to provide a periodic or continuous flow of air or a combination of other suitable gases to the patient.

As noted earlier, some patients may be required to use medical devices at home to maintain or improve their health. In addition, government agencies and insurance companies may have an interest in ensuring that such patients use these machines properly, as these entities typically spend millions of dollars on this type of healthcare. Even so, it may be difficult to monitor some of these medical devices, particularly those that are older.

As a solution, a method and system for monitoring a medical device to ensure usage compliance by a patient are described herein. The method can include the steps of monitoring one or more parameters associated with the operation of the medical device, comparing the monitored parameters with one or more predetermined operational profiles and generating a signal that is indicative of whether the medical device is being used by the patient in substantial conformance with a predetermined usage pattern assigned to that patient. In one arrangement, the use of the medical device by the patient requires voluntary actions on the part of the patient to be in substantial conformance with the predetermined usage pattern.

As such, a patient's use of a medical device can be easily monitored and reported to any suitable entity. Through this monitoring, a patient can be apprised of the progress of his/her treatment, and improvements can be executed where needed.

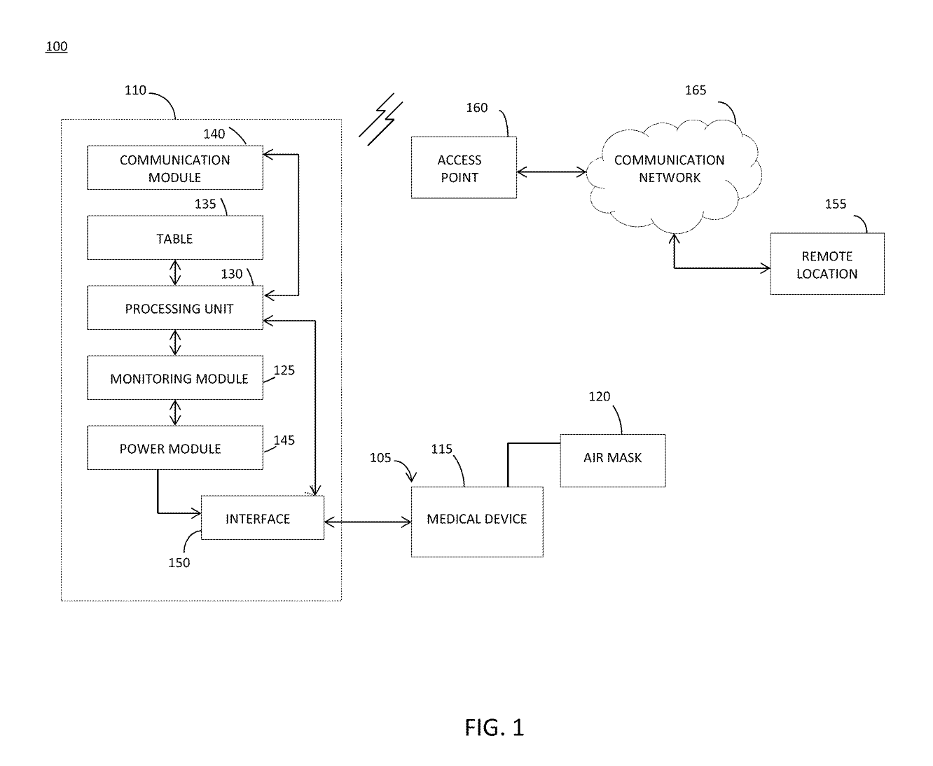

Referring to FIG. 1, an example of a medical device monitoring system 100 is shown. In one arrangement, the system 100 can include a medical device 105 and a medical device monitor 110. In this example, the medical device monitor 110 can be a component that is separate from the medical device 105, meaning that the monitor 110 can be physically separated from the device 105 or the device 105 can operate under normal conditions without the use of the monitor 110. In one arrangement, the medical device 105 can be a breathing assistance machine 115 having an air mask 120, like a continuous positive airway pressure (CPAP) machine or sleep apnea machine. Although presented as a breathing assistance machine 115, the medical device 105 is certainly not limited to this type of unit. In fact, the medical device monitor 110 is not limited to monitoring medical devices, as the monitor 110 can be used to monitor other suitable electronic devices.

In one arrangement, the medical device monitor 110 can include a monitoring module 125, a processing unit 130, a table 135 and a communication module 140. The medical device monitor 110 can also include a plug or power module 145 and an interface 150. The processing unit 130 can be communicatively coupled to the monitoring module 125, the table 135 and the communication module 140, and the monitoring module 125 can be communicatively coupled to the power module 145. Further, the power module 145 can be electrically coupled to the interface 150, which can be electrically coupled and (optionally) communicatively coupled to the medical device 105. Although shown as separate entities, any of the components described above can be integrated into a smaller number of units. For example, the monitoring module 125 and/or the table 135 can be part of the processing unit 130.

The monitoring module 125 can be configured to monitor one or more parameters associated with the operation of the medical device 105. As will be explained later, the processing unit 130 can generate signals that provide information that is related to the operation of the medical device 105. In addition, through the communication module 140, the medical device monitor 110 can communicate such signals to a remote location 155, where any suitable processing or analysis of these signals can be performed. A remote location in this context can refer to any location where suitable equipment may be kept to process or analyze the signals received from the medical device monitor 110. The remote location 155 may be positioned at a location that is physically removed from an area housing the medical device 105 and the medical device monitor 110, or alternatively, it may be in the same physical area of the device 105 and monitor 110.

In one particular but non-limiting arrangement, the communication module 140 of the medical device monitor 110 can be communicatively coupled to an access point 160. There are numerous configurations for this connection to be achieved, such as through Wi-Fi, cellular, ZigBee, POTS or Ethernet. The access point 160 can be communicatively coupled to a communication network 165, which can be any network or group of networks that can enable wide-ranging communications, such as the Internet. The communication network 165 can also be communicatively coupled to the remote location 155. It is understood that other suitable scenarios can be employed to enable the transfer of signals between the medical device monitor 110 and the remote location 155. For example, as an option, the medical device monitor 110 can be configured to transmit signals directly to the remote location 155. As another option, the medical device 105 can be configured to communicate signals to the remote location 155, either directly or through suitable facilitators, as will be explained later. In either arrangement, any combination of wired and wireless connections can be used to conduct these communications.

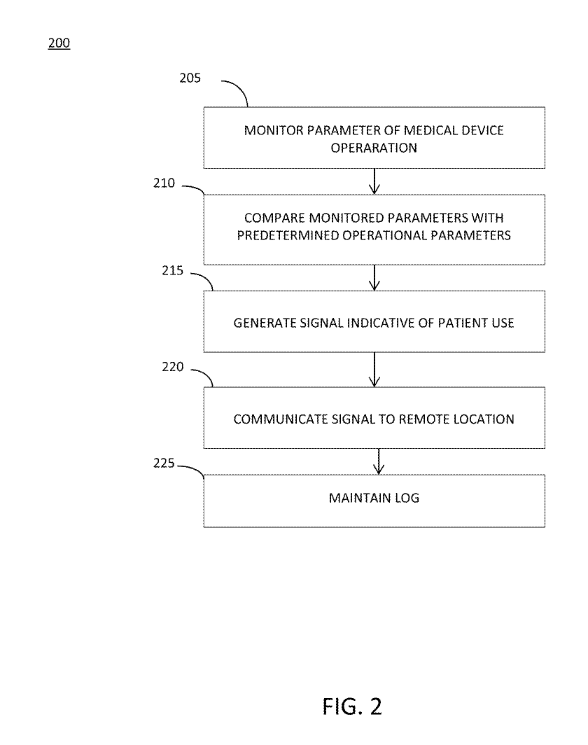

Referring to FIG. 2, a method 200 for monitoring a medical device to ensure usage compliance by a patient is shown. To describe the method 200, reference will be made to the components of FIG. 1, although it is understood that the method 200 can be practiced using other suitable components and systems. Also, the method 200 may be practiced with other additional steps not shown in FIG. 2 or may be practiced with a fewer number of steps as shown in FIG. 2. The method 200 is not necessarily limited to the chronological order established in FIG. 2, either.

At step 205, one or more parameters associated with the operation of a medical device can be monitored, and at step 210, the monitored parameters can be compared with one or more predetermined operational parameters. At step 215, a signal can be generated that is indicative of whether the medical device is being properly used by the patient. The signal can then be communicated to a remote location, as shown in step 220. As an option, at step 225, a log that contains information related to the monitoring of the parameters can be maintained.

An example of the method 200 will now be presented. As mentioned earlier, the medical device monitor 110 may include a power module 145. The power module 145 may be configured to engage a wall outlet or some other power source. In addition, the medical device 105 may include a power module (not shown), which can engage the interface 150 of the monitor 110. As such, the medical device 105 can receive its power through the medical device monitor 110.

The monitoring module 125, through its interaction with the power module 145, can monitor one or more parameters associated with the operation of the medical device 105. The phrase “monitor[ing] one or more parameters” is defined as observing, recording or detecting one or more values, including individual values or ranges of values. In one arrangement, these parameters can be related to the power consumption of the medical device 105. For example, the parameters can include an on/off state, amperes, voltage, real power or apparent power. The monitoring process can occur continuously or periodically (or even randomly), depending on the necessity of collecting information about the operation of the medical device 105. Of course, the monitoring module 125 can be used to monitor other suitable parameters associated with the medical device 105, including those that are not related to electrical consumption. In any event, the monitoring module 125 can send the monitored parameters to the processing unit 130.

Once it receives the monitored parameters, the processing unit 130 can compare these parameters with one or more predetermined operational profiles, which may be stored in the table 135. A “predetermined operational profile” is defined as a known value or a known range of values (whether individually or in combination with other known values or known ranges of values) that are associated with the operation of a device at a particular setting or a particular mode. In one embodiment, the predetermined operational parameters can be related to the power consumption of the medical device 105, thereby providing a convenient basis for comparison. For example, the predetermined operational profiles can include a predetermined on/off state, a predetermined value or range of values of amperes, a predetermined value or range of values of voltage, a predetermined value or range of values of real power or a predetermined value or range of values of apparent power. It is understood, however, that the predetermined operational parameters are certainly not limited to these particular examples, as other suitable criteria can serve as a predetermined profile. These predetermined values or ranges of values can be adjusted or calibrated to ensure that such values or ranges of values accurately correspond to a particular use or mode of the device being monitored. For example, such adjustments or calibrations can be conducted if a different patient uses the monitored device or the current patient's condition changes.

Consider the following as an example. The medical device 105, as described earlier, may receive its power through the medical device monitor 110. The operational parameter, in this example, may be the number of amperes (“amps”) being supplied to the medical device 105. Proper operation of the medical device 105 by a patient may cause a certain average amount of amps to be drawn over the course of a period of time. In particular, if the air mask 120 of the medical device 110 is properly engaged with the face of a patient over the course of a certain number of hours, then the average amount of current drawn over that time can provide a reflection of that appropriate use. Any number of factors can influence the number of amps consumed during the operation of the medical device 105. For example, the back air pressure generated by the air mask 120 being engaged with the patient's face may affect the electrical operation of a fan (not shown) or other components supplying the forced air. As another example, a heater (not shown) may be activated to warm the forced air or a humidifier (not shown) may be initiated to moisten the air when the air mask 120 is engaged with the patient's face, either of which can increase the number of amps used.

The use of the air mask 120 in this example may also affect the values for voltage, real power, apparent power or other suitable parameters. In addition, the monitoring module 125 can simply monitor the on/off state of the medical device 105. In fact, any combination of these parameters can be monitored, including just one of them or all of them. As noted earlier, other suitable parameters can be monitored, as well.

Continuing with the amps example, once measured, the processing unit 130 can compare the value of the measured amps to a known value in the table 135. If the measured value meets or exceeds the known value (or even if it does not meet or exceed the known value), then the processing unit 130 can generate a signal that is indicative of whether the medical device 105 is being used by the patient in substantial conformance with a predetermined usage pattern or a prescribed therapy that is assigned to that patient. This process can include the processing unit 130 (or some other suitable component of the medical device monitor 110) taking active steps to determine whether the patient is in compliance or merely collecting data and forwarding it to a remote location for processing such that the determination of compliance is made by a component other than the medical device monitor 110. The phrase “substantial conformance with a predetermined usage pattern [or prescribed therapy] assigned to a patient” includes exact conformance with the usage pattern or prescribed therapy or deviations from that usage pattern or prescribed therapy that do not warrant a notice to the patient to improve his/her conformance.

As an example, a predetermined usage pattern or a prescribed therapy can be a treatment that has been assigned to the patient by a medical provider to maintain or improve the health of the patient. In another example, the use of the medical device 105 by the patient may require voluntary actions on the part of the patient to be in substantial conformance with the predetermined usage pattern or the prescribed therapy. For example, the patient may be suffering from sleep apnea, and a doctor may prescribe the use of the breathing assistance machine 115 to improve the patient's condition. As part of this therapy, it may be necessary for the patient to put on and wear the air mask 120 for continuous periods of time. The processes described above can be used to determine whether the patient has been in compliance with his/her treatment.

In one arrangement, a log can be maintained that contains information relating to the monitoring of the parameters. For example, the processing unit 130 can record data that shows whether the patient has been in conformance with the assigned treatment plan, and the data can be stored in the table 135 or some other component. This data can include the actual values of the monitored parameters (e.g., amps, voltage, etc.), periods of time that have been monitored and indications as to whether the patient's use of the medical device 105 is in accordance with the treatment plan for those periods of time. Alternatively (or in addition to), this data can be recorded at a location other than the medical device monitor 110.

As part of the monitoring process, the signal that is indicative of whether the medical device 105 is being used by the patient in substantial conformance with the predetermined usage pattern or prescribed therapy can be communicated to the remote location 155, as described above. For example, the processing unit 130 can signal the communication module 140, and the module 140 can take the steps necessary to forward the collected data to the remote location 155. The communication of the data can occur instantaneously and continuously, or the communication module 140 can forward the relevant information in accordance with a predetermined schedule. Once received at the remote location 155, the information can be processed in any suitable manner at the remote location 155 or some other suitable setting.

Any suitable entity can analyze the received information. For example, medical personnel may review the information to determine whether the patient has been properly following his/her treatment program. If not, the patient may be contacted in an effort to improve the patient's conformance. These results may also be forwarded to other organizations, like insurance companies or government agencies, which may also take corrective action to improve the patient's treatment.

The examples described above may be particularly useful if the medical device 105 is a legacy device. In this arrangement, the medical device monitor 110 can be a retrofitted component that increases the functionality of the legacy medical device. A “legacy device,” as used in this context, is defined as a device that lacks the capability of monitoring certain parameters and reporting the results of that monitoring to another component or a remote location. It is understood, however, that the medical device 105 is certainly not limited to being a legacy device. In fact, the examples described herein are not even limited to use with a medical device. Other components, like manufacturing devices, vehicular monitoring systems or educational devices can be monitored and analyzed in accordance with the above description.

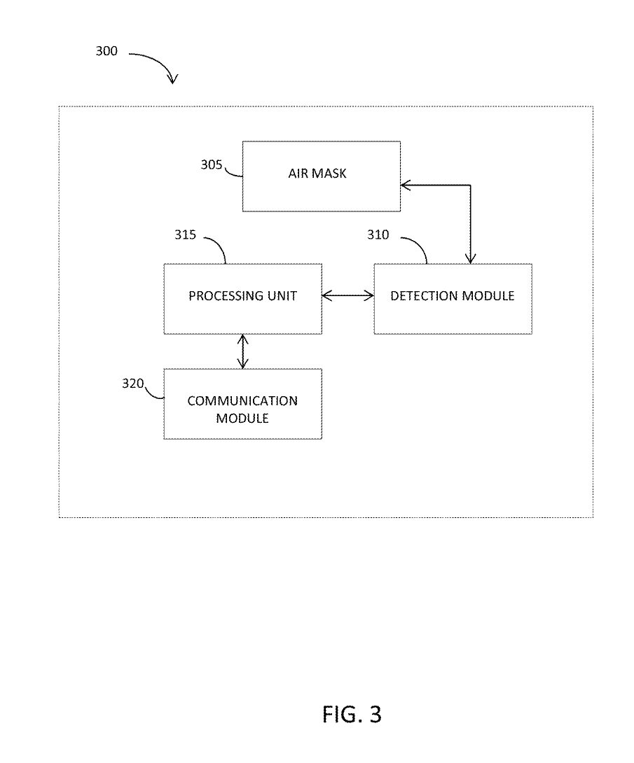

As opposed to a legacy system, some devices may already include all the elements necessary to monitor and convey information about a patient's use of that device. Referring to FIG. 3, an example of such a system is illustrated. Here, a medical device monitoring system 300 is shown in block diagram form. The medical device monitoring system 300 can form part of a prescribed therapy. In one arrangement, the system 300 can include an air mask 305, which can be configured to engage the face of a patient to permit forced air to be provided to a patient. The system 300 can also include a detection module 310, a processing unit 315 and a communication module 320.

In one arrangement, the detection module 310 can be operable to monitor one or more thresholds to determine whether the air mask 305 is engaged with the face of the patient. In addition, the processing unit 315 can be operable to generate a signal that is indicative of whether the patient is in substantial conformance with the prescribed therapy based on the frequency that the air mask 305 is engaged with the face of the patient.

For example, one of the thresholds used to determine whether the air mask is engaged with the face of the patient can be a back air pressure that is generated by the air mask 305. In particular, if the patient places the air mask 305 over his/her face, a back air pressure may be generated, which can be measured and may be higher than the back air pressure generated if the air mask 305 is not engaged with the patient's face. The detection module 310 can monitor this pressure and can signal the processing unit 315 if the pressure meets or exceeds (or fails to meet or exceed) an air pressure threshold. The thresholds can be adjusted or calibrated to ensure correspondence with a particular setting or mode of operation. For example, if a different patient uses the air mask 305, a different threshold may be used to indicate proper usage of the system 300.

Similar to that previously described, the processing unit 315 can compare the measured parameter(s) with a predetermined threshold. In this case, the processing unit 315 can compare the measured air pressure with a predetermined air pressure to determine whether the air mask 305 is engaged with the face of the patient. Further, the processing unit 315 can generate a signal that is indicative of the use of the system 300. In this example, the processing unit 315 (or some other suitable component) can generate a signal that indicates whether the patient is in substantial conformance with a predetermined usage pattern or prescribed therapy based on the frequency that the air mask 305 is engaged with the face of the patient. Specifically, the generated signal can provide data that is reflective of the actual use of the air mask 305 by the patient. This level of processing at the system 300 and, hence, the amount of information conveyed by the generated signal can be set to any suitable configuration, similar to that previously described. Some may find it better to have the system 300 perform most of the processing of the collected information, while others may prefer to have more of it done at a remote location.

In response to the generation of such signals, the processing unit 315 can cause the communication module 320 to forward the signal to, for example, a remote location (not shown here). Once received at the remote location, the information concerning the patient's treatment can be analyzed, and if necessary, the patient can be contacted to improve his/her treatment plan.

An example has been presented here in which air pressure associated with the use of an air mask has been employed to determine whether a patient has been properly using a medical device. It is understood, however, that other parameters may be monitored or detected for this purpose. For example, the air mask 305 may be outfitted with sensors (not shown) that can detect the engagement of the air mask 305 with the patient's face. As another example, the air mask 305 may include one or more thermometers (not shown) that can be configured to measure the temperature changes associated with the engagement of the mask 305 with the face of the patient. In yet another example, a carbon dioxide detector can be integrated with the air mask 305 or some other part of the device to detect levels of carbon dioxide from the patient wearing the air mask 305. A similar configuration can be arranged to detect water vapor from the patient, which can also be used to determine proper engagement of the air mask 305. There are numerous other examples that are applicable here, as well.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

While various embodiments of the present invention have been described above, it should be understood that they have been presented by way of example only, and not limitation. It will be understood by those skilled in the relevant art(s) that various changes in form and details may be made therein without departing from the spirit and scope of the invention as defined in the appended claims. Accordingly, the breadth and scope of the present invention should not be limited by any of the above-described exemplary embodiments, but should be defined only in accordance with the following claims and their equivalents.

1. A method for monitoring a medical device to ensure usage compliance by a patient, comprising:
monitoring one or more parameters associated with the operation of the medical device;
comparing the monitored parameters with one or more predetermined operational profiles corresponding to a prescribed therapy assigned to that patient; and
generating a signal that is indicative of whether the medical device is being used by the patient in substantial conformance with a predetermined usage pattern assigned to that patient.

2. The method according to claim 1, wherein the use of the medical device by the patient requires voluntary actions on the part of the patient to be in substantial conformance with the predetermined usage pattern.

3. The method according to claim 1, further comprising communicating the signal indicative of whether the medical device is being used by the patient in substantial conformance with the predetermined usage pattern to a remote location.

4. The method according to claim 1, wherein the monitored parameters include:
an on/off state;
amperes;
voltage;
real power; or
apparent power.

5. The method according to claim 4, wherein the predetermined operational profiles include:
a predetermined on/off state;
a predetermined value or range of values of amperes;
a predetermined value or range of values of voltage;
a predetermined value or range of values of real power; or
a predetermined value or range of values of apparent power.

6. The method according to claim 1, further comprising maintaining a log that contains information relating to the monitoring of the parameters.

7. The method according to claim 1, wherein the medical device is a breathing assistance machine.

8. The method according to claim 1, wherein monitoring the parameters, comparing the monitored parameters and generating the signal is performed at a power device that provides power to the medical device.

9. The method according to claim 1, wherein the power device is separate from the medical device.

10. A medical device monitor, comprising:
a monitoring module that is configured to monitor one or more parameters associated with the operation of a medical device;
a table that is configured to store predetermined operational parameters corresponding to a prescribed therapy; and
a processing unit, wherein the processing unit is communicatively coupled to the monitoring module and the table and is configured to:
compare the monitored parameters with the predetermined operational parameters stored in the table; and
generate a signal that is indicative of whether the medical device is being used by a patient in substantial conformance with a predetermined usage pattern assigned to that patient.

11. The medical device monitor according to claim 10, further comprising a communication module that is configured to communicate the signal indicative of whether the medical device is being used by the patient in substantial conformance with the predetermined usage pattern to a remote location.

12. The medical device monitor according to claim 10, the use of the medical device by the patient requires voluntary actions on the part of the patient to be in substantial conformance with the predetermined usage pattern.

13. The medical device monitor according to claim 10, wherein the parameters that are monitored by the monitoring module include:
an on/off state;
amperes;
voltage;
real power; or
apparent power.

14. The medical device monitor according to claim 10, wherein the predetermined operational parameters include:
a predetermined on/off state;
a predetermined value or range of value of amperes;
a predetermined value or range of values of voltage;
a predetermined value or range of values of real power; or
a predetermined value or range of values of apparent power.

15. The medical device monitor according to claim 10, wherein the processing unit is further configured to maintain a log that contains information relating to the monitoring of the parameters.

16. The medical device monitor according to claim 10, wherein the medical device is a breathing assistance machine.

17. The medical device monitor according to claim 10, further comprising a plug that is configured to couple to a power outlet to permit the medical device monitor to provide power to the medical device.

18. A medical device monitor to ensure compliance with a prescribed therapy assigned to a patient, comprising:
a monitoring module that is configured to monitor one or more parameters associated with the operation of a medical device, wherein the medical device forms part of the prescribed therapy, wherein the one or more parameters correspond to the prescribed therapy;
a processing unit that is coupled to the monitoring module, wherein the processing unit is configured to generate a signal that is indicative of whether the medical device is being used by the patient in substantial conformance with the prescribed therapy; and
a power module that is configured to provide power to the medical device.

19. The medical device monitor according to claim 18, wherein the medical device monitor is a component that is separate from the medical device.

20. The medical device monitor according to claim 19, wherein the medical device is a legacy device and the medical device monitor is a retrofitted component that increases the functionality of the legacy medical device.

21. A medical device monitoring system for ensuring compliance with a prescribed therapy assigned to a patient, comprising:
a medical device that forms part of the prescribed therapy; and
a medical device monitor that provides power to the medical device, wherein the medical device monitor comprises:
a monitoring module that is configured to monitor one or more parameters associated with the operation of the medical device;
a table that is configured to store predetermined operational parameters corresponding to a prescribed therapy assigned to the patient; and
a processing unit, wherein the processing unit is communicatively coupled to the monitoring module and the table and is configured to:
compare the monitored parameters with the predetermined operational parameters stored in the table; and
generate a signal that is indicative of whether the medical device is being used by the patient in substantial conformance with the prescribed therapy assigned to that patient.

22. The medical device monitoring system according to claim 21, wherein the medical device is a breathing assistance machine that includes an air mask and the prescribed therapy requires that the patient wear the air mask.

23. A medical device monitoring system that forms part of a prescribed therapy for a patient, comprising:
an air mask that is configured to engage the face of a patient to permit forced air to be provided to the patient, wherein when engaged with the patient, an air pressure is generated by the air mask;
a detection module that is configured to monitor one or more thresholds to determine whether the air mask is engaged with the face of the patient; and
a processing unit that is configured to generate a signal that is indicative of whether the patient is in substantial conformance with the prescribed therapy based on the frequency that the air mask is engaged with the face of the patient.

24. The medical device monitoring system according to claim 23, further comprising a communications module that is operable to transmit the generated signal to a remote location.

25. The medical device monitoring system according to claim 23, wherein one of the thresholds used to determine whether the air mask is engaged with the face of the patient is the air pressure generated by the air mask.

26. A method for determining whether a patient is in substantial conformance with a predetermined usage pattern assigned to that patient, comprising:
measuring an air pressure that is generated by an air mask that is intended to be engaged with the face of the patient as part of the predetermined usage pattern;
comparing the measured air pressure with a predetermined threshold to determine whether the air mask is engaged with the face of the patient; and
generating a signal that is indicative of whether the patient is in substantial conformance with the predetermined usage pattern based on the frequency that the air mask is engaged with the face of the patient.
